(19) United States
(12) Patent Application Publication
(10) Pub. No.: US 2025/0235096 A1
(43) Pub. Date: Jul. 24, 2025

(54) APPARATUS FOR WAVEFRONT ABERROMETRY

(71) Applicant: XENON OPHTHALMICS INC., Belleville, NJ (US)

(72) Inventors: ZESHAN ALI KHAN, BELLEVILLE, NJ (US); STEVE SUSANIBAR, Morris Plains, NJ (US); Viachaslau LOSIK, Hemau (DK); Siarhei SIDAROVICH, Novoselye, Minsk district (BY)

(73) Assignee: XENON OPHTHALMICS INC., Belleville, NJ (US)

(21) Appl. No.: 18/855,560

(22) PCT Filed: Apr. 11, 2023
(86) PCT No.: PCT/US2023/018205

§ 371 Date: Oct. 9, 2024

Related U.S. Application Data
(60) Provisional application No. 63/362,866, filed on Apr. 12, 2022.

(51) Int. Cl. A61B 3/10; A61B 3/14

(52) U.S. Cl. CPC A61B 3/1015; A61B 3/145

(57) ABSTRACT

An aberrometer system comprises a headset frame configured to be worn on the face of a user and an optical assembly which is coupled to the headset frame to be in alignment with one of the user's eyes. The optical assembly comprises an off-axis aberrometer channel and an on-axis eye tracking channel with an on-axis eye imaging camera and an off-axis subchannel with a display and a variable power lens assembly that can to adjust the apparent distance of a displayed image as viewed through the eyepiece. The optical assembly can be removably or movably mounted to the headset for positioning in front of either eye.

### CROSS-REFERENCE TO RELATED APPLICATIONS

This application claims priority to U.S. Provisional Patent Application No. 63/362,866, filed Apr. 12, 2022, the entire contents of which is incorporated by reference.

### FIELD OF THE INVENTION

The present invention is related to improvements in systems for visual testing and imaging, and in particular a headset mounted aberrometer system.

### BACKGROUND

Eye care is an important part of overall health and many specialized systems have been developed to allow ophthalmologists to examine a person's eyes. Many of these devices are expensive, limiting their availability. They are also bulky, often requiring a dedicated table or mounting a special stand. The size, weight, and general ungainliness of these devices also can require dedicated space in a doctor's office to be reserved for that equipment.

One device used for eye testing is an aberrometer, a diagnostic device that uses a laser and wavefront analysis to measure refractive aberrations of the eye for evaluating issues such as nearsightedness, farsightedness and astigmatism, as well as more complex visual defects. Conventional aberrometers are table top devices that can be expensive and heavy to move around. A patient must sit in front of the aberrometer system and press their head against a specially placed face bar or mask to position their eyes in front of the optical window. A doctor can control the system and monitor the results through a separate computer interface.

However, patients who have mobility limitations may not be able to easily make an office visit or be physically able to position themselves as required for examination using a particular optical tool. This can limit the ability to provide comprehensive eye examinations to these patients. Likewise, due to bulk and expense, it may be difficult or impossible to bring a variety of these specialized eye examination systems to a patient who is not able to travel to the doctor's office.

It is known to provide certain types of eye testing equipment in a portable form that can be integrated into a head mounted system. However, these systems can be limited. There is a need for a portable and inexpensive system for performing aberrometry in a variety of environments and that has an architecture that is well suited for use in a headset mounted implementation.

### SUMMARY

An improved aberrometer assembly suitable for integration a VR-style headset mount and that addresses the deficiencies with prior systems is disclosed herein.

In an embodiment, the aberrometer system comprises a headset frame configured to be worn on the face of a user. A first optical assembly is coupled the headset frame so the central optical axis of the optical assembly is in alignment with an eye of the user when the headset frame is worn. The optical assembly comprises a first beam splitter that defines an eye tracking channel having an eye tracking axis that substantially co-linear with the central optical axis. The first beam splitter also defines an aberrometer channel with an aberrometer optical axis that is off axis from the central optical axis.

The eye tracking channel comprises an IR imager with an imaging plane, an eyepiece, and a camera lens. An IR illuminator is provided to illuminate the eye of a user wearing the headset. The the eyepiece and camera lens configured to focus IR light reflected from the first eye of the user onto an imaging plane of the imager. The eye tracking channel further has a second beam splitter defining an off-axis display subchannel having a display optical axis. The display subchannel comprises adjustable display optics and an electronic display. The adjustable display optics are configured to relay an image output on the display to the eyepiece via the second beamsplitter so the image can be viewed by the user wearing the headset.

The aberrometer channel comprises an array camera, a plurality of lenses in alignment with the aberrometer optical axis and a third beam splitter defining a light source subchannel. The light source subchannel comprises a laser that emits the light used during aberrometry. The aberrometer channel is configured to direct laser light from the light source subchannel along the aberrometer optical axis to the first beamsplitter and to relay to the array camera laser light reflected by the first eye of the user when the user is wearing the headset frame and directed by the first beamsplitter into the aberrometer channel so the wavefront can be captured by the array camera.

In an embodiment, the adjustable display optics comprises a liquid lens with an electronically controlled spherical diopter power and a display lens assembly. Adjusting the spherical diopter power can change an apparent distance of an image output by the display as viewed through the eyepiece. Varying the liquid lens to move the apparent distance of an image far away can help the user put their eye into an unaccommodated state for imaging in the aberrometry process. The eye tracking camera can be used to verify that the eye is open and the user is looking straight ahead.

In an embodiment, the IR illuminator comprises a ring of IR emitters circling the central optical axis and on a side of the first beamsplitter furthest from the eyepiece. This placement allows for full IR illumination of the eye and so that substantially all of the IR light entering the eyepiece is reflected light from the user.

In the aberrometer channel, the light source subchannel can further comprise a focusing lens assembly between the laser and the beam splitter. The plurality of lenses in the aberrometer channel can comprise two set of lenses. The third beam splitter can be placed between the two lens sets. In an embodiment, to reduce the total length of the aberrometer channel along a single axis, the aberrometer channel can further comprise a diagonal which can be placed adjacent the third beam splitter and is operative to redirect the aberrometer optical axis.

In an embodiment, the aberrometer optical axis and display optical axis are each substantially normal to the central optical axis. These two axes can also be substantially coplanar.

Any of the embodiments of the first optical assembly can be removably mounted to the headset frame and can be switched from being mounted in front of one eye or the other.

Any of the embodiments of the first optical assembly can also be mounted to the headset frame so they can be moved from a first position in front of a first eye to a second position in front of a second eye of the user. In an embodiment, the first optical assembly is mounted so it can be rotated from the first to the second position, and also thereby moving any component similarly mounted to the headset frame in front of the second eye. In another embodiment, the first optical assembly is slidably mounted so it can be moved from the first to the second position by lateral translation. An adjustable light baffle can be coupled to the first optical assembly so that when the first optical assembly is in the first position the baffle covers the second eye region and when the first optical assembly is in the second position the baffle covers the first eye region.

Any of the embodiments of the first optical assembly can also be integrally mounted to the headset frame. Where only one optical assembly is provided, the system can be used with either eye by configuring the headset so it can be use right-side up or upside down. In one embodiment, the headset frame has two nose channels, one on top and one on the bottom. An adaptor can be provided to cover up the unused nose channel and provide a top surface of the headset that can extend to the user's forehead.

With any of the embodiments of the first optical assembly, the headset frame can have an opening in front of the other eye. A user wearing the headset frame for aberrometry of one eye can see through the opening with the other eye so they can focus on a distant object, simplifying bringing the eye being measured into an unaccommodated state needed for accurate aberrometry.

Along with the first optical assembly, second optical assembly can be coupled to the headset in front of the second eye region. The second optical assembly can be removably mounted or integral to the headset and can be in its own housing or the first and second optical assemblies can be mounted within a common outer housing. The mounting can allow the user of the headset to switch which eye the first and second optical assemblies are positioned in front of, such as by rotation or translation. In an embodiment, the second optical assembly is an eye tracking and display system, such as the eye tracking channel component of the first optical assembly. In a further embodiment, the second optical assembly is the same as the first optical assembly, thereby allowing aberrometry of both eyes to be performed without having to modify the headset. Other types of optical assemblies could be used as the second optical assembly.

The aberrometer, eye tracking, and display channels can be controlled by a computer system to vary the stimulus shown on the displays, vary the power of the liquid lens, perform eye tracking and monitoring the a user's eye, and to control initiate the aberrometry process to measure refractive aberrations of the eye.

### DESCRIPTION OF THE DRAWINGS

Further features and advantages, as well as structure and operation of various embodiments are disclosed in detail below with references to the accompanying drawings in which:

FIG. 1 is a schematic diagram of an improved aberrometer system;

FIG. 2A is a simplified diagram of an aberrometer implementing the architecture of the aberrometer system of FIG. 1;

FIG. 2B is a cross-sectional rendering of a particular embodiment of the aberrometer channel of FIG. 2A;

FIGS. 3A-3D are embodiments of a headset-mounted aberrometer system;

FIGS. 4 and 5 are illustration of an aberrometer system movably mounted to a headset frame;

FIGS. 6A and 6B show an optical layout embodiment of an aberrometer channel of the system of FIG. 1; and

FIG. 7 shows an optical layout embodiment for the eyepiece and camera lens assembly of the system of FIG. 1.

### DETAILED DESCRIPTION

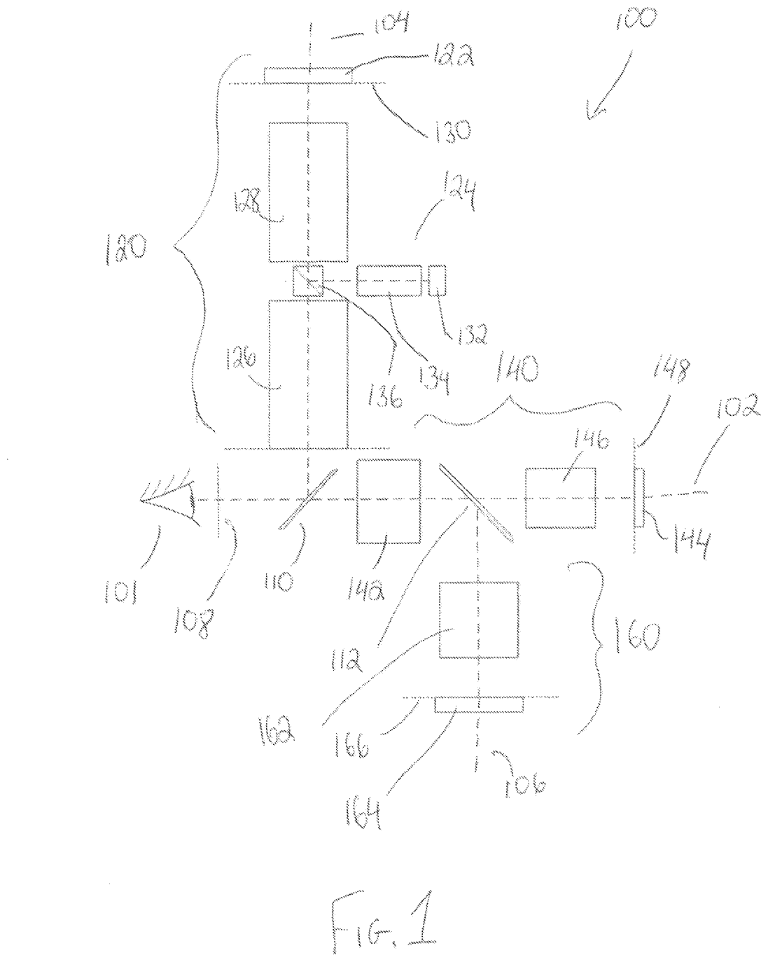

FIG. 1 is a schematic diagram of an improved aberrometer system 100 that can be used to measure refractive aberrations of the eye. The system 100 can be formed within a compact and lightweight housing that can be attached to a headset that is worn on a person's face. System 100 has a central optical axis 102 that leads to a plurality of optical channels. A front or first beam splitter 110 separates the optical system into two primary channels: (i) an aberrometer channel 120 having an aberrometer optical axis 104, and (ii) an eye tracking channel 140. The eye tracking channel 140 is located along the primary optical axis 102 so that when system 100 is in use this channel will be directly in front of the user's eye 101. In order to capture the wave front image of the user's eye 101 via the aberrometer, the user's pupil must be in an unaccommodated state and not be physically offset from the central axis 102 by more than a small distance. This placement of the eye tracking channel along the primary axis 102 allows for optimal tracking of the eye pupil and to ensure that the user is looking straight ahead and that their eye is open when aberrometry is performed. It also allows for the eye tracking channel to be designed more easily with a wide field of view.

The eye tracking channel 140 comprises an eyepiece 142, an imaging camera 144, and a camera lens assembly 146 which is configured to focus incoming light onto the imaging plane 148 of the camera 144. The camera 144 can be a CMOS or other sensor with a rolling or global shutter. Various cameras known to those of skill in the art for eye tracking applications can be used. One suitable camera is a Basler daA3840-45 um camera that has a mono CMOS rolling sensor with 4K UHD resolution, a USB data interface, and a housing of about 20 mm (L)×29 mm (W)×29 mm (W) in size.

A second beam splitter 112 is positioned between the eyepiece 142 and camera lens assembly 146 and leads to a display channel 160 having a display optical axis 106 that is off axis from the central optical axis 102, such as by substantially 90 degrees, although other angles, such as 45 or 30 degrees could be used. The display channel 160 comprises adjustable display optics 162 and a display 164. Display 164 can be a conventional flat panel display, such as an LED, LCD, micro mirror or OLED display, with sufficient resolution to present adequate images to a user as part of aberrometry testing. In an embodiment, the display is a small display, such as between 30 to 40 mm and of the type commonly used in smart watch applications. One suitable display is the Kingtech model PV13904PY24G-C1 AMOLED display with a panel size of about 35 mm and a resolution of 454×454 pixels.

An illumination source 111 for the eye tracking camera 144 is provided to produces IR light, such as in a wavelength region of from 800 nm to 900 nm. The camera lens assembly 146 is configured for use in these wavelengths. Light from source 111 that is reflected from the eye 101 is received by eyepiece 142 which forms an image of the eye 101 on an intermediate image plane between the second beam splitter 112 and camera lens assembly 146. Camera lens assembly 146 conjugates the intermediate image plane with the camera sensor plane 148. The optical performances of camera lens assembly 146 and the eyepiece 142 are matched.

Illumination source 111 is positioned to provide sufficient illumination of the eye for eye tracking and imaging purposes. In an embodiment, illumination source 111 comprises a plurality of IR LEDs (850 nm wavelength) which are placed between the eye plane 108 and the first beam splitter 110 in a position that will illuminate the eye 101 without blocking the eye 101. The amount of illumination required can vary depending on factors including the sensitivity of the camera 144. In an embodiment, the LEDs are configured to provide an irradiance of approximately 225 W/m². The adjustable display optics 162 operate as relay optics to transfer an image output on display 164 to the second beam splitter 112 to allow the user to see the display (through eyepiece 142). Adjustable display optics 162 comprises a relay lens and a liquid lens that can be driven electronically to adjust the spherical power provided, such as across a range+10 to −10 diopters or other diopter range as appropriate. A suitable lens is the Optotune model EL-16-40-TC. The relay lens is configured to correct for color aberration within a range of visual wavelengths, such as 480 nm to 640 nm. The second beam splitter 112 is configured to reflect visible light from the display channel and to pass IR wavelength light to the eye tracking camera 144.

In an embodiment, a technician or doctor can control the spherical power of the liquid lens in the display optics 162, such as through a remote computer interface coupled to system 100, to allow for a rapid and large diopter change to blur the image seen on the display. The blurring of the image is an important aspect of preparing the user's eye for wave front capturing. The liquid lens The liquid lens can also be autonomously controlled by software operative to change the spherical power and that monitors images from the eye tracking channel to determine when the user is looking straight ahead and not blinking so that the aberrometry process can be started

The aberrometer channel 120 comprises a Shack-Hartmann array camera 122, a light source channel 124 and a set of lenses 126, 128 which are configured to capture the reflected wave front from the user's eye 101 and provide it to the imaging plane 130 of the array camera 122. Light source channel 124 comprises and laser diode 132 and a focusing lens assembly 134 to collimate the laser light. A third beam splitter 136 is placed along the aberrometer optical axis 104 and is used to convey the light laser light into the aberrometer channel along its optical axis 104. The laser diode 132 can be a low power laser diode, such as 1 mw or less, and that generates 680 nm red laser light. The beam splitter 136 can be a precision cube beam splitter to minimize wave front distortions.

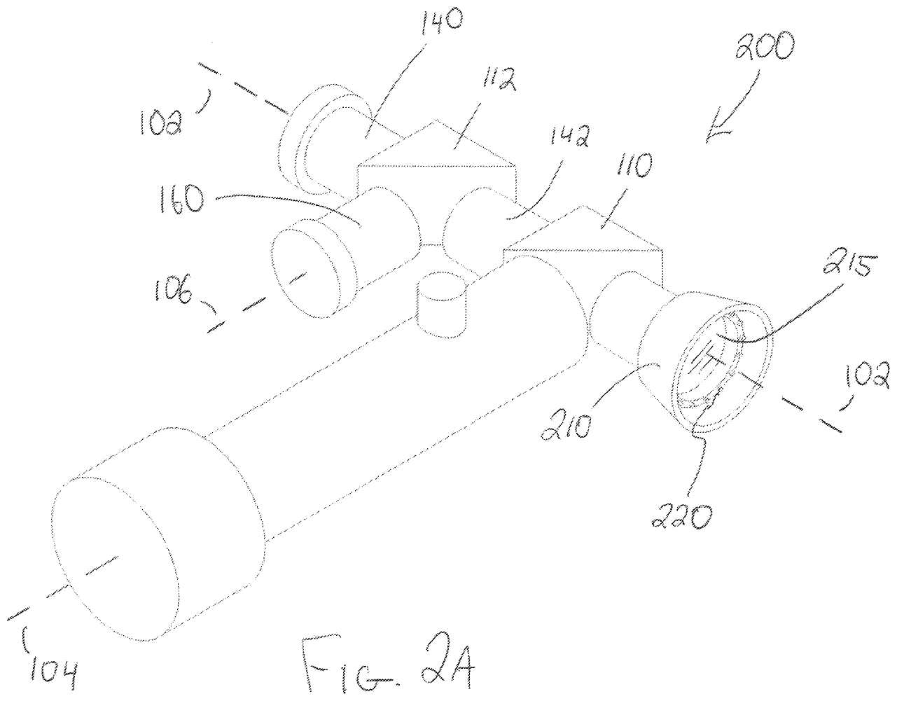

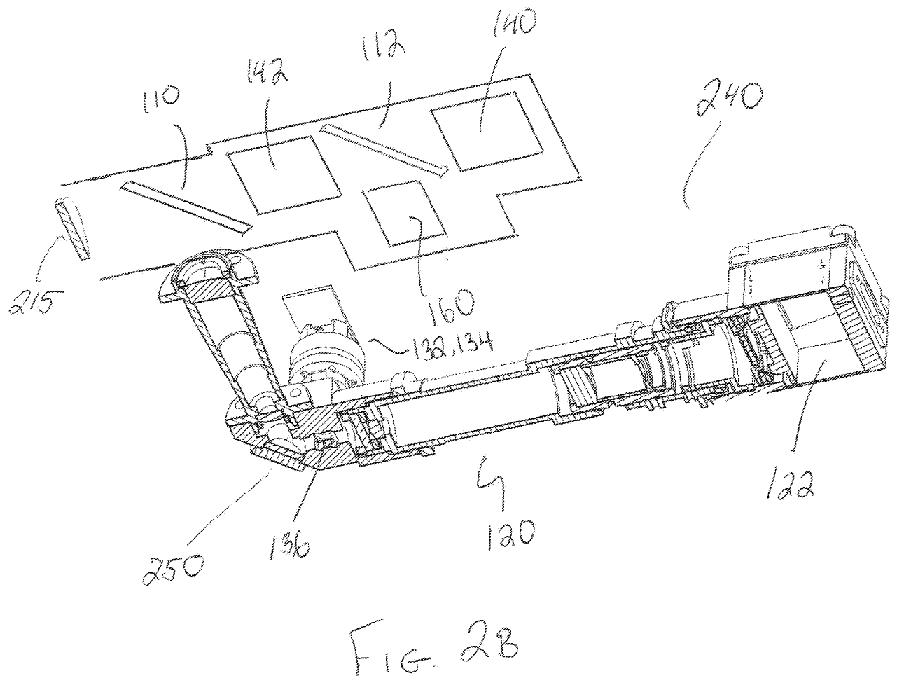

FIG. 2A is a simplified diagram of an aberrometer 200 implementing the architecture of the aberrometer system 100 of FIG. 1 and showing the aberrometer, eye tracking, and display channels 120, 140, 160 along with the first and second beam splitters 110, 112 and eyepiece 142. In this configuration, the first and second beam splitters 110, 112 are oriented so that the aberrometer optical axis 104 and display optical axis 106 are substantially co-linear. However, in alternative embodiments, other relative rotational orientations between these components can be used depending on how the aberrometer 200 is packaged.

An eye cup 210 extends forward off the first beam splitter 110 for use in helping to position a user's eye along the central optical axis 102 and to reduce stray light entering the system. A glass cover plate 215 can be used to seal the internals of the system 200. The illuminators 111 can be configured as a circumferential ring of a plurality of IR LEDs 220 surrounding the central optical axis 102. Ring 220 can be placed in a variety positions. In FIG. 2A, ring 220 is placed around the cover plate 215 and along the inner periphery of the eye cup 210. The LEDs can be mounted on an IR reflective substrate to increase efficiency of the illumination.

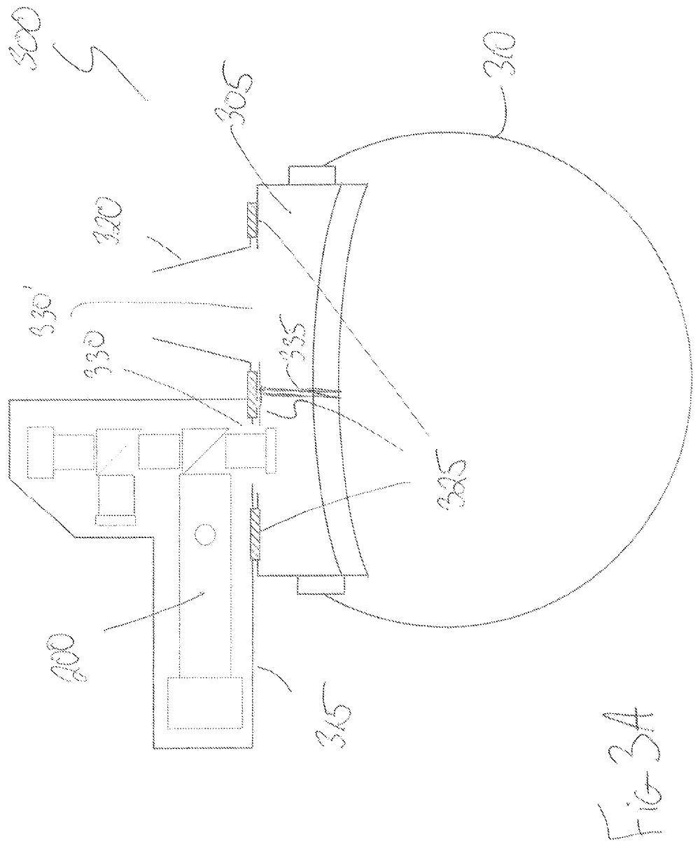

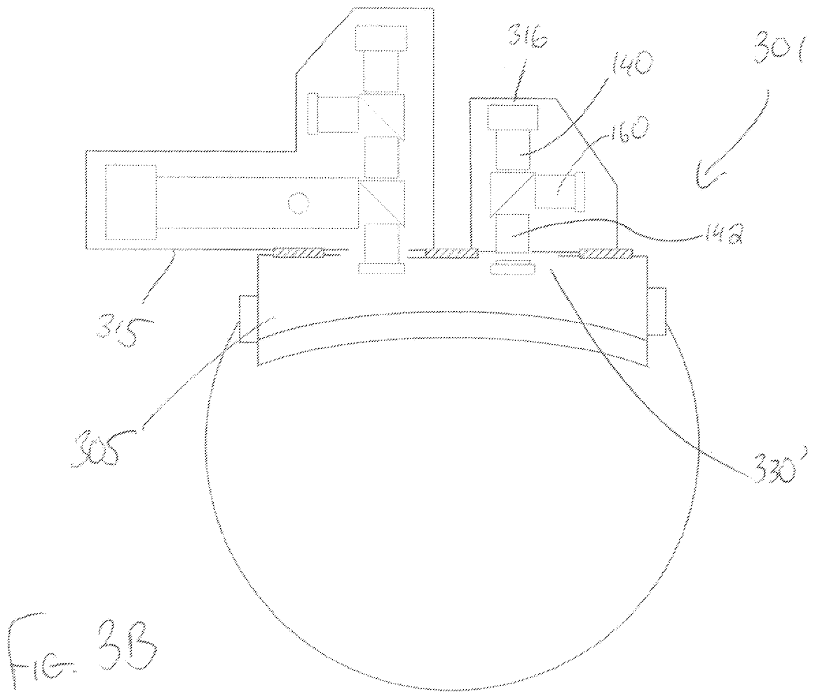

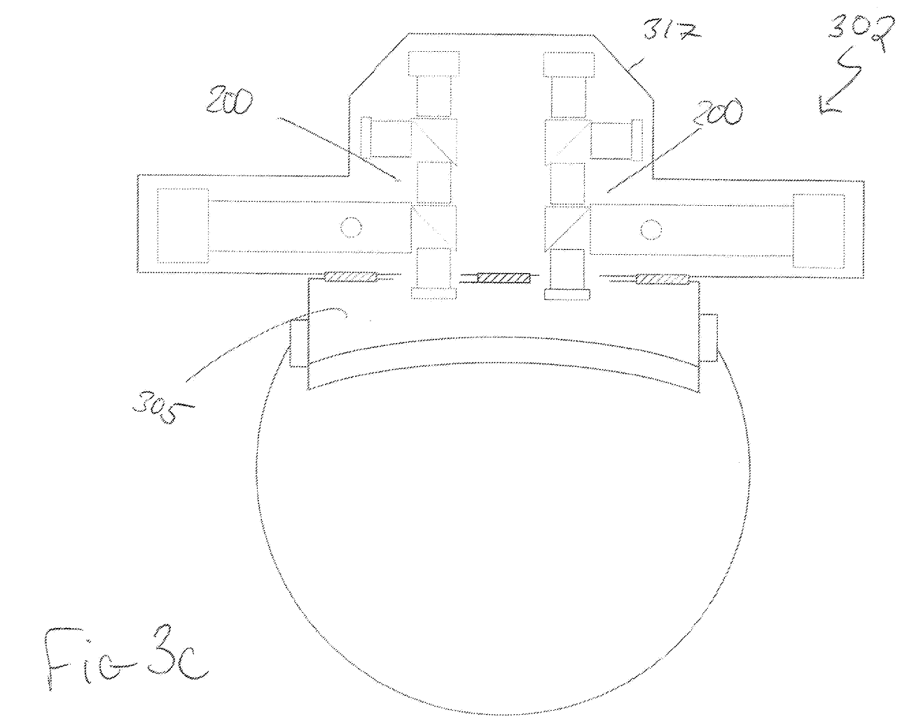

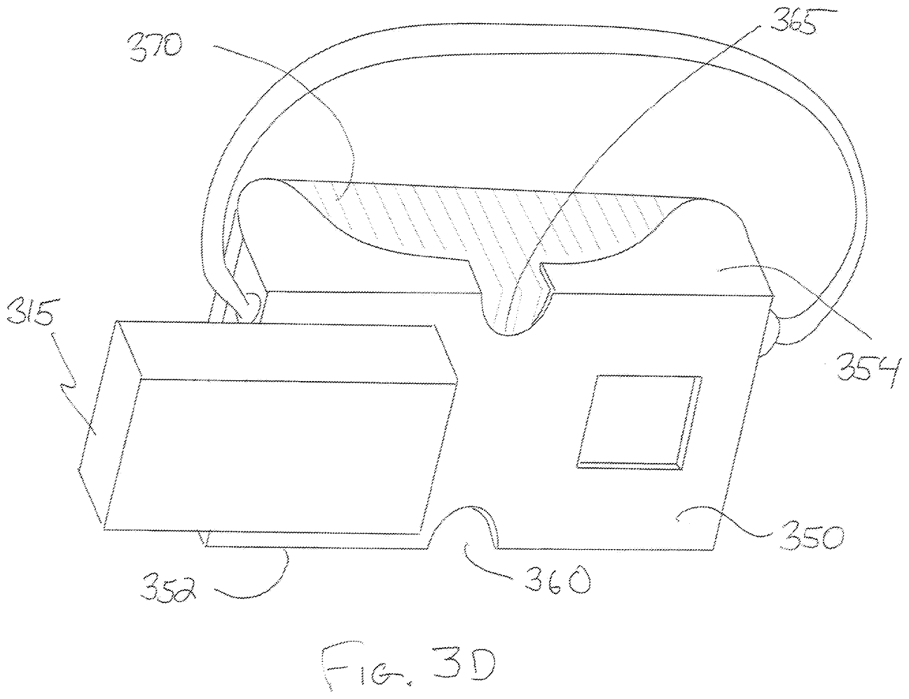

Advantageously, having the eye tracking channel 140 directly in line with the central optical axis 102, components of the camera lens assembly 146 can be placed close to the beam splitter 112 without interfering with the reflected image from the display channel 160. This allows for a larger field of view in the eye tracking camera. In addition, having the aberrometer channel 120 and display channel 160 off-axis from the central optical axis 102 also advantageously allows the length of the optical assembly 110 along the central optical axis 102 to be short relative to the width of the other channels. This form factor advantageously allows the assembly 110 be integrated with a wearable headset, such as discussed with respect to FIGS. 3A-3D with the bulk of the weight positioned to the left and right of the eyepiece instead of extending outward from the eyepiece and the user's face. As a result, the center of mass of the system remains close to the user's face so that the overall weight of system 100 is largely downward and strain on a user's neck is reduced.

FIG. 2B shows a cross-sectional rendering of a particular embodiment 240 of the aberrometer channel 120 which further includes a diagonal 250 operative to redirect the aberrometer optical axis 104 aberrometer channel. Also shown in a schematic view is a representative placement of the imaging and display channels 140, 160, the eyepiece 142, and the first and second beam splitters 110, 112. Other configurations of these components may also be used.

In an embodiment, diagonal 250 is a mirror configured to reflect the laser light frequency and is optically flat to introduce minimal distortion to the wave front. In an embodiment, the lenses, beam splitter and diagonal of the aberrometer channel are configured so that introduced wave front deviation is within 1/10 λ of the illumination. The use of diagonal 250 in the configuration 240 of FIG. 2B allows the overall length of the aberrometer channel 120 to be reduced with an increase in width. Such a configuration can allow for a more convenient packaging of the system, e.g., for mounting on a wearable headset as discussed further below. In this embodiment, the diagonal 250 is substantially 90 degrees. However, other angles, such as 45 or 30 degrees, can be used depending on the form factor desired.

Advantageously, the improved aberrometer system 100 can be configured to be small and light enough to be mounted within a housing coupled to a headset that can be worn on a person's face. FIG. 3A is an illustration of a headset-mounted aberrometer system 300. System 300 comprises a headset frame 305 with supporting straps 310 configured so a user can wear the headset with the frame 305 securely positioned in front of the user's eyes. The headset frame has first and second eye regions, wherein when the headset is worn the first eye region is in front of the user's first eye and the second eye region is in front of the user's second eye.

In this embodiment, the headset frame 305 has left and right openings 330, 330′ which are positioned to be in front of the user's eyes when the headset system 300 is worn. Alternatively, a single opening can be provided and which is in front of both of the user's eyes. A housing 315 contains the aberrometer 200 and is mounted on one side of the headset frame 305 so that the central optical axis 102 passes through one opening 330. In FIG. 3A, the aberrometer 200 is positioned in the headset so that the aberrometer channel 120 extends laterally. Other configurations are also possible. For example, the aberrometer 200 can be mounted in the housing so that the aberrometer channel 120 extends upwards, downwards, or in another direction.

The housing 315 may be integral with the headset frame 305 or removably coupled thereto, such as with coupling hardware 325. Mechanisms for coupling an optics module to a headset and which can be adapted to the present use are disclosed in U.S. Pat. No. 11,504,000 entitled “Ophthalmologic Testing Systems and Methods”, the entire contents of which are expressly incorporated by reference. The housing 315 or aberrometer 200 within the housing can be mounted to allow the position of the central optical axis 102 to be laterally adjustable so it can be positioned in alignment with a user's eyes. A vertical adjustment may also be provided.

Various manual or automated adjustable mounting mechanisms known to those of skill in the art can be used, such as a track mounting driven by linear actuators or a stepper motor. In an embodiment with an electronically controllable adjustment mechanism, the eye tracking camera can be used to adjust the position of the central optical axis 102 relative to the headset frame 305, either under control of an operator who can view the images output from the eye tracking camera, e.g., on a remote computer, or automatically using software to analyze the images returned from the camera, to position the optical axis 102 correctly relative to the user's eye.

During use of this embodiment, when a first eye of a user is in alignment with the aberrometer for imaging, the user's other eye is aligned with the opening 330′ in the headset frame. The user can look through this opening 330′ to a distant point far away from the headset in order to bring the non-imaged eye into an unaccommodated state. Once the non-imaged eye is relaxed, the imaged eye will also be relaxed and thus in a state that is ready to be imaged. Blinders 320 can be provided in front of opening 330′ to help the user focus attention on the distant point.

In order to properly image the eye, the pupil needs to be as dilated as possible and therefore stray light from opening 330′ should be blocked. In an embodiment a divider 335 is positioned within the headset frame 305 between openings 330 and 330′. Divider 335 can extend sufficiently in the headset frame 305 so that when the frame is worn the divider will contact the user's face. Divider 335 can be made of a confirmable material that will adjust to the contours of the user's face to reduce the possibility of light leakage. Alternatively, instead of blinders 320, the opening 330′ can be completely covered or omitted entirely although this might make it more difficult for a user to bring their eyes to an unaccommodated state, even with the use of the adjustable liquid lens in the display optics to change the apparent distance of a displayed image viewed by the imaging eye.

According to a further embodiment, the aberrometer assembly 200 can moved to allow imaging of either of the user's eyes. In one configuration, the housing 315 is removably mounted to the headset frame 305 and can be connected in front of either of the eye openings 330, 330′. After one eye is imaged, the housing 315 can be disconnected from the headset frame 305 and reattached in front of the other eye opening and reoriented as necessary. Blinders 320 or other covering can also be removably mounted and switched from one eye opening to the other as needed.

Other embodiments are variations of the configuration of FIG. 3A and wherein other optical assemblies can be removably attached to or integrated as part of the headset frame 305. FIG. 3B shows a headset system 301 which is a variation of the system 300 of FIG. 3A but with a second optical assembly 316 placed over the second opening 330′. System 316 as illustrated comprises the eye tracking channel 140 with eyepiece 142 and the display channel 160 portions of the system 100 but without the aberrometer channel 120. Such a configuration can be advantageously allow the user to be shown the same image in both eyes and provide a virtual image with a faraway apparent distance to allow the user to put both eyes in an unaccommodated state even if a distant real object to view is not available. By using the same optics in assembly 316 as in assembly 316, the components such as the display and liquid lens state, can be controlled in an identical manner and the resulting images will optically match. The dual display system will also allow images to be presented in virtual 3D. In alternatives, system 316 could comprises only the display channel 160 (with the eyepiece 142) or only the eye tracking channel 140.

In a particular configuration, a modular system is available for the headset frame where an operator can attach an aberrometer housing 315 to one eye opening and then select from a cover, blinders 325, an imaging and eye tracking system in housing 316, or even a second aberrometer and attach that to the opposing eye opening on the headset frame. Other components could also be attached for use in different types of eye examinations.

Instead of being removably attached and swappable from one side to the other, one or more of the aberrometer assembly and the alternate eye cover, blinders, eye tracking system, or second aberrometer system can be integrally attached to the headset 305. For example, a system can be provided with the aberrometer system in housing 315 integral to the headset with the second eye component removable and replaceable with one or more of the components discussed above.

In a further variation 302 shown in FIG. 3C, left and right aberrometer systems 200 can be combined in a single housing 317 that can be removably attached or integrated with the headset frame 305. Mounting systems known to those of ordinary skill in the art can be used to allow the left and right aberrometers 200 to be symmetrically moved towards or away from each other in order to adjust the intraocular distance as needed for a given user.

Yet a further embodiment is shown in FIG. 3D. In this embodiment, a housing 315 with an aberrometer system 200 therein is mounted in front of one of the eye regions of a headset 350. The headset 350 has a bottom 352 and a top 354. The bottom 352 has a centrally located nose channel 360. When the headset 350 is worn with the top 354 upwards, the nose channel 360 will fit over the user's nose. The top 354 has a second centrally located nose channel 365. A removable adapter 370 is fitted to the top 354 of the housing 315 to fill in the second nose channel 365 and to provide a cover for the top of the headset that can rest against the user's forehead. To perform aberrometry of the other eye, the headset can be rotated so bottom 352 is facing upwards. The adapter 370 is removed from the top surface and placed in the bottom surface to cover the first nose channel.

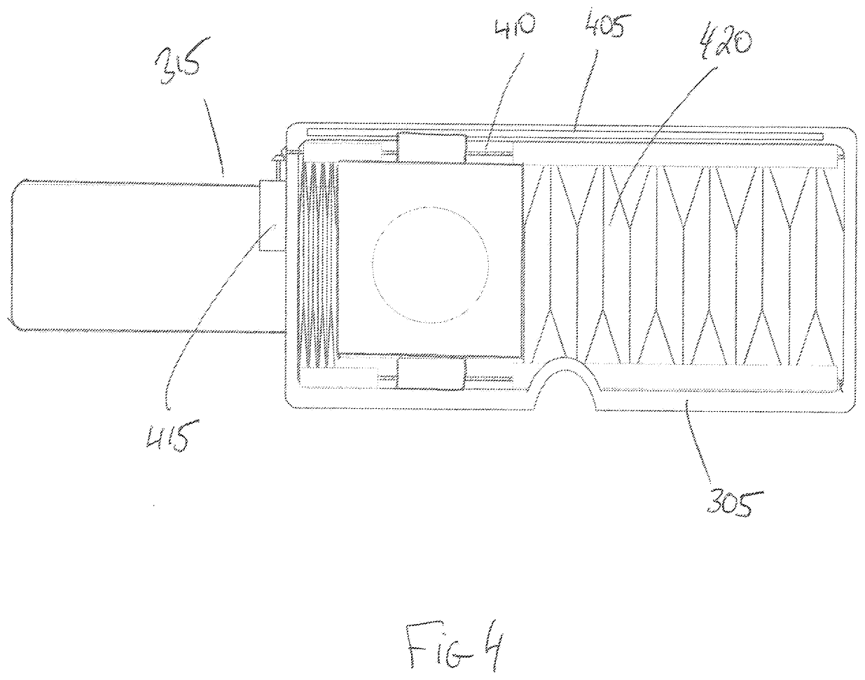

FIG. 4 is an illustration of a system 400 with a single aberrometer 200 in housing 315 slidably mounted to the headset frame 305 to provide for laterally adjustment in front of one eye and for repositioning of the central optical axis 104 of the aberrometer in front of either eye. The aberrometer housing 315 is mounted on a rail system 405 and coupled to a threaded shaft 410 that is driven by a motor 415. Rotation of the shaft 410 causes lateral motion of the housing 315. To block stray light from entering the system, an adjustable light blocking baffle can be used. For example, an opaque fan-fold material 420 can be affixed to the housing 315 so it can expand and contract as the housing 315 is moved.

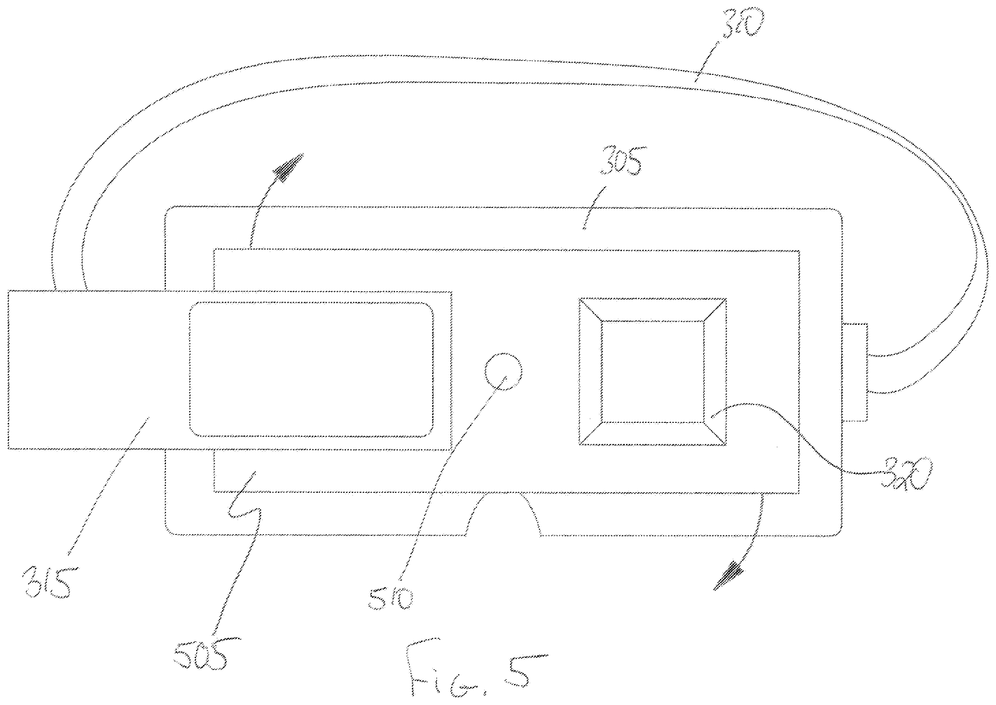

In yet a further embodiment, the housing 315 with the aberrometer 200 within can be rotatably mounted to the front of the headset frame 305. FIG. 5 shows such a rotatable configuration in which the housing 315 is attached to a mounting plate 505 which is pivotally mounted to the front of the headset frame 305 at a pivot point 510. Alternative eye hardware, such as blinder 320 or other optical equipment can be mounted to the headset frame as well.

Detents or other mechanisms (not shown) can be used to secure the mounting plate 505 in a fixed position. Various rotatable mounting mechanisms known to those of ordinary skill in the art can be used. In one embodiment, the rotatable mounting assembly comprises a spring that urges the mounting plate 505 against the headset frame. To switch the position of the aberrometer and blinder 320 from one eye to the other, the mounting plate 505 can be pulled outward from the headset frame, rotated 180 degrees and released. Instead of a spring, a screw mechanism can be used and that is configured to hold the mounting plate 505 rigidly against the headset frame 305 when it is tightened and to allow for rotational motion when listened. Latches or other similar mechanisms (not shown) can be used to secure the mounting plate 505 in position on the headset frame 305 during eye imaging operations.

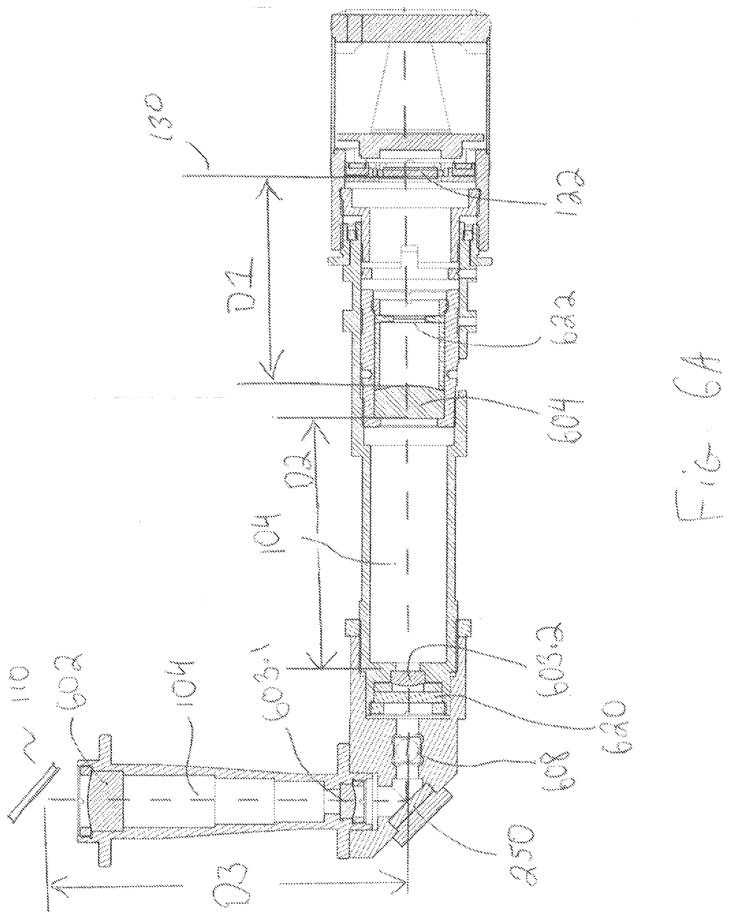

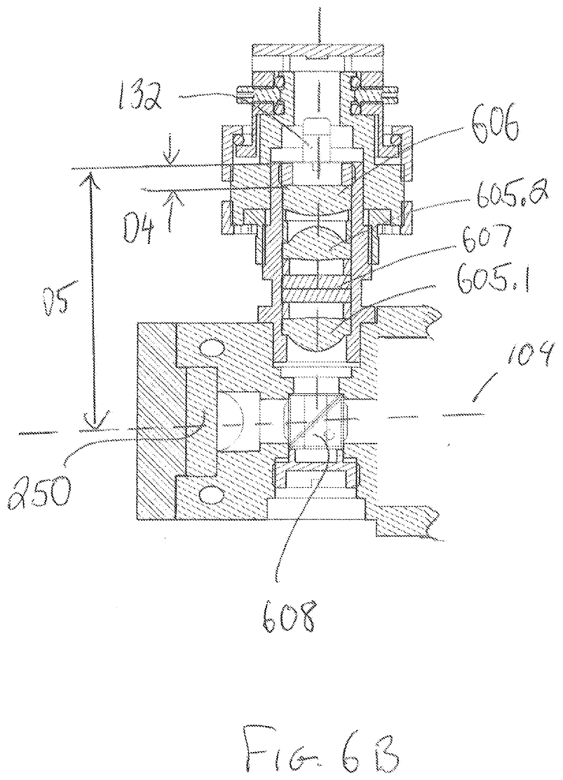

Returning to FIG. 1, the lenses 126, 128 in the aberrometer channel 120 are configured so that the laser wave front provides a good wave front plane with only a small deviation from the plane wave front geometry so the reflected wave front from the user's eye can be accurately captured by the array camera 122. After this data is captured, software can be used to extract values that can be translated into a baseline, objective prescription for corrective glasses. One specific embodiment of aberrometer channel 120 is shown in FIGS. 6A and 6B

FIG. 6A shows the optical layout of the aberrometer channel 240 of FIG. 2B along the aberrometer optical axis 104. FIG. 6B shows the optical layout of an embodiment of the laser diode 132 and focusing lens assembly 134. Turning to FIGS. 6A and 6B, following the first beam splitter 110, the aberrometer comprises lenses configured to operate with the wave front analyzer camera 122 to provide wave front accuracy measurements up to 5th order of Zernike with a range of aperture of measurements from 2 to 8 mm.

The wave front sensor 122 is proceeded by an aspherical lens 604 for capturing collimated wave fronts at a distance D1. Another aspherical lens 603.2 is located a distal position of D2 away from the aspherical lens 604. A beam splitter 608 is used to direct light from an off-axis laser diode onto the aberrometer optical axis 104. This light is used to illuminate the user's eye 101. An aspheric lens 603.1 is positioned between the beam splitter 608 on-axis with the first beam splitter 110 and is also positioned within a fixed distance D3 of a larger aspherical lens 602 that is adjacent to the first beam splitter 110. A polarizer 620 is included in the optical path, such as between the cube beam splitter 608 and lens 603.2. An aperture 622 is placed after lens 604 and in front of the wave front camera 122. In an embodiment, aspheric lens 602 has a 25 mm diameter and 0.4 numerical aperture. Aspheric lenses 603.1 and 603.1 are 6.33 mm diameter and lens 604 is 25 mm in diameter with a 0.3 numerical aperture.

Turning to FIG. 6B, the laser focusing lens assembly 134 comprises three aspherical lenses 605.1, 605.2, and 606, with lens 606 a distance D4 from the laser diode 123. A glass aperture 607 is situated between lenses 605.1 and 605.2. These lenses are optimized to match with the wave front deviation within 1/10 λ of the illumination frequency. The total distance from the laser diode 132 to the aberrometer optical axis 104 is D5. A suitable light source comprises a quasi-single mode 680 nm vertical-cavity surface-emitting laser (VCSEL) with linear polarized emission. This laser is particularly favorable for its red wavelength which provides high resolution in addition to having a small spot size.

In a particular embodiment, the lenses in the aberrometer channel can be configured to provide a compact system with the element distances substantially as set forth in Table 1 below:

TABLE 1

| | | | |
|---|---|---|---|
| D1 | ~38 mm | 37.74 | mm |
| D2 | ~48 mm | 48.09 | mm |
| D3 | ~79 mm | 79.14 | mm |
| D4 | ~12 mm | 11.8 | mm |
| D5 | ~41 mm | 41.03 | mm |

In operation, light from the laser diode 132 is focused by the aspherical lenses 606 and 605.2 on the glass aperture 607. The aperture 607 forms point source beam which is collimated by aspherical lens 605.1 and then reflected by the cubic beam splitter 608 into the aberrometer optical channel 104. The beam passes through aspherical lenses 603.1 and 602 where it is reflected by the first beam splitter 110 to the eye 101 being tested. Light reflected from the eye 101 is redirected by the first beam splitter 110 into the aberrometer channel 102, passes through lenses 602, 603.1, beam splitter 608 and continues through additional aspherical lenses 603.2 and 604, finally landing on the imaging surface 130 of the wave front camera 122.

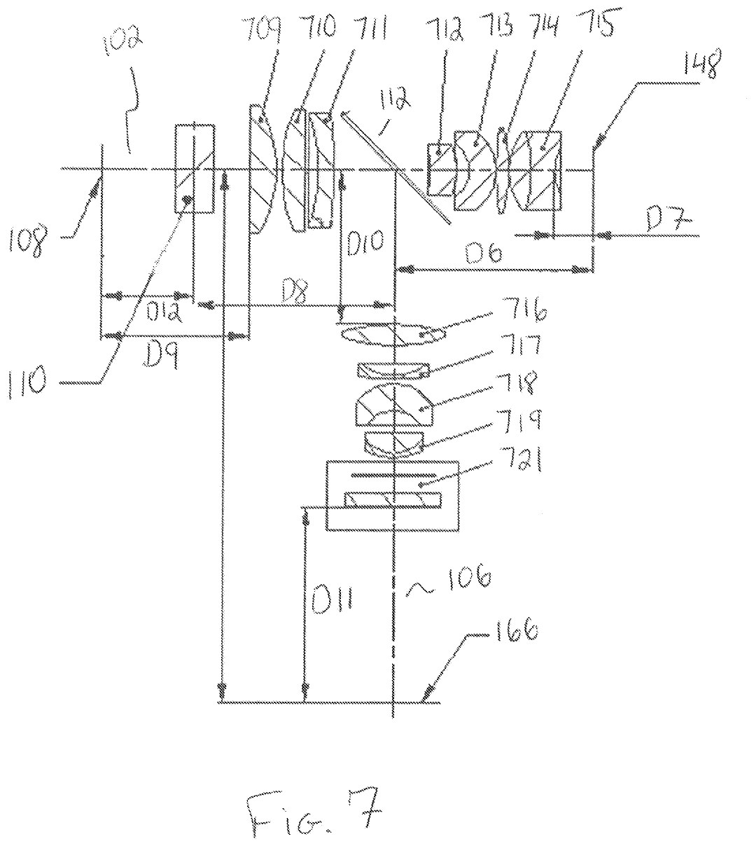

FIG. 7 shows an embodiment of an optical layout for the eyepiece 142 and camera lens assembly 146 of the eye tracking channel 140 and of the display optics 162 of the display channel 160. Eyepiece 142 comprises three spherical lenses 709, 710, and 711 coupled to each other to reduce distortion and stray reflections from the first beam splitter 110. Camera lens assembly 146 operates as a relay lens and in this embodiment comprises spherical lenses 712, 713, a thin spherical lens 714, and a spherical doublet lens 715. These lenses are configured to project a clear, undistorted image onto the sensor plane 148 of the eye tracking camera 144. The lenses are configured to provide a 20 mm field of view at the eye plane 108.

In an embodiment of FIG. 7, the display optics 162 comprises a relay lens that includes a spherical field lens 716 and a four elements lens group with a spherical lens 717, spherical lens 718, and a spherical doublet lens 719. The optical performance if this relay lens group is matched to that of the lenses in the eyepiece 142. A liquid lens 721 is positioned directly in front of the display image plane 166. The display channel 160 can be configured to provide a field of view of at least 40 degrees (from Eye side) and 4 mm diameter of pupil.

Lens 715 is a distance D7 from the imaging plane 148 and the imaging plane 148 is a distance D6 from the second beam splitter 112. The first and second beam splitters 110, 112 are distance D8 apart. Lens 708 in the eyepiece 142 is a distance D9 from the eye plane 108. In the display channel, the relay lens 716 is a distance D10 from the beam splitter 112. Liquid lens 721 is a distance D11 from the display image plane 166. The first beam splitter 110 is a distance D12 from the eye plane 108. In a particular embodiment, the lenses in the eyepiece 142, camera lens assembly 146, and display optics are configured to provide a compact system with the element distances substantially as set forth in Table 2 below:

TABLE 2

| | | | | |
|---|---|---|---|---|
| D6 | ~52 | mm | 51.75 | mm |
| D7 | ~10 | mm | 10.00 | mm |
| D8 | ~52 | mm | 52.46 | mm |
| D9 | ~38 | mm | 38.41 | mm |
| D10 | ~40 | mm | 39.98 | mm |
| D11 | ~52 | mm | 51.5 | mm |
| D12 | ~19 | mm | 19 | mm |

In this specific embodiment, the 5-element camera lens assembly has NA substantially equal to 0.1, a FOV substantially equal to 20 mm, and a weight of about 30 g. The display optics projection lens is a 5-element group providing a pupil diameter Dp of substantially 6 mm and a FOV of substantially 40 degrees with a weight of about 25 g. The total length of the display channel is about 138 mm from the central optical axis 102 to the display image plane 166.

A position adjustment mechanism for the aberrometry optical module can be included to allow for lateral adjustment to allow alignment with the eye of different users that have different intraocular spacings. When two optical modules are provided on the headset, the adjustment mechanism can be configured to move the assemblies symmetrically towards or away from each other to adjust the intraocular spacing. Various adjustment mechanisms known to those of ordinary skill in the art can be used for these purposes.

Various aspects, embodiments, and examples of the invention have been disclosed and described herein. Modifications, additions and alterations may be made by one skilled in the art without departing from the spirit and scope of the invention as defined in the appended claims.

1. An aberrometer system comprising:
a headset frame configured to be worn on the face of a user, the headset frame having a front, a back, and first and second eye regions, wherein when the headset frame is worn by a user the back of the frame is adjacent a face of the user and the first and second eye regions are adjacent and in general alignment with a first eye and second eye, respectively of the user;
a first optical assembly having a central optical axis and coupled to the headset frame so the central optical axis is in alignment with the first eye region and extends outwards from the front of the headset frame;
the first optical assembly comprising:
a first beam splitter defining an eye tracking channel having an eye tracking axis substantially co-linear with the central optical axis and an aberrometer channel having an aberrometer optical axis that is off axis from the central optical axis;
an infrared (IR) illuminator configured to emit light to illuminate the first eye of the user when the user is wearing the headset frame;
the eye tracking channel comprising an IR imager with an imaging plane, an eyepiece, and a camera lens, the eyepiece and camera lens configured to focus IR light reflected from the first eye of the user onto an imaging plane of the imager;
the eye tracking channel further comprising a second beam splitter defining an off-axis display subchannel having a display optical axis, the display subchannel comprising adjustable display optics and an electronic display, the adjustable display optics configured to relay an image output on the display to the eyepiece via the second beamsplitter for viewing by the first eye of the user when the user is wearing the headset frame; and
the aberrometer channel comprising an array camera, a plurality of lenses in alignment with the aberrometer optical axis and a third beam splitter defining a light source subchannel comprising a laser, the aberrometer channel configured to direct laser light from the light source subchannel along the aberrometer optical axis to the first beamsplitter and to relay to the array camera laser light reflected by the first eye of the user when the user is wearing the headset frame and directed by the first beamsplitter into the aberrometer channel.

2. The system of claim 1, the adjustable display optics comprising a liquid lens with an electronically controlled spherical diopter power and a display lens assembly, wherein adjustment of the spherical diopter power can change an apparent distance of an image output by the display as viewed through the eyepiece.

3. The system of claim 1, the IR illuminator comprising a ring of IR emitters circling the central optical axis and on a side of the first beamsplitter furthest from the eyepiece.

4. The system of claim 1, the light source subchannel further comprising a focusing lens assembly between the laser and the beam splitter.

5. The system of claim 1, the plurality of lenses in the aberrometer channel comprising a first plurality of lenses and a second plurality of lenses, the third beam splitter between the first plurality of lenses and the second plurality of lenses.

6. The system of claim 4, the aberrometer channel further comprising a diagonal adjacent the third beam splitter and operative to redirect the aberrometer optical axis.

7. The system of claim 1, wherein the aberrometer optical axis is substantially normal to the central optical axis and the display optical axis is substantially normal to the central optical axis.

8. The system of claim 1, wherein the aberrometer optical axis and the display optical axis are substantially coplanar.

9. The system of claim 1, wherein the first optical assembly is removably mounted to the headset frame in front of the first eye region and can be removed from the headset frame and removably mounted in front of the second eye region.

10. The system of claim 1, wherein the first optical assembly is movably mounted to the headset frame and is positionable on the headset frame through one of translation and rotation relative to the headset from a first position with the central optical axis within the first eye region and a second position with the central optical axis in alignment with the second eye region.

11. The system of claim 1, wherein the first optical assembly is positionable from the first position to the second position via translation, the system further comprising an adjustable light baffle coupled to the first optical assembly, wherein when the first optical assembly is in the first position the baffle covers the second eye region and when the first optical assembly is in the second position the baffle covers the first eye region.

12. The system of claim 1, wherein the first optical assembly is integral with the headset frame; the headset frame having a bottom with a first nose channel and a top with a second nose channel and is configured to be wearable by the user in a first orientation with the central optical axis in alignment with the first eye of the user and with the first nose channel over a nose of the user and in a second orientation with the central optical axis in alignment with the second eye of the user and the second nose channel over the nose of the user.

13. The system of claim 1, the headset frame having an opening in the second eye region, wherein when wearing the headset frame the user can see through the opening with the second eye.

14. The system of claim 13, further comprising a second optical assembly coupled to the headset in front of the second eye region and having a second central optical axis, wherein when the headset frame is worn by the user, the second central optical axis is in alignment with the second eye of the user.

15. The system of claim 14, wherein the second optical assembly comprises a second IR illuminator, a second eye tracking channel having a second eye tracking optical axis in substantial alignment with the second central optical axis and comprising a second eyepiece, second camera lens, and a second imager, the second eyepiece and second camera lens configured to focus IR light reflected from the second eye of the user onto an imaging plane of the second imager.

16. The system of claim 15, the second optical channel further comprising a fourth beam splitter in the second central optical axis between the second eyepiece and the second camera lens and defining a second off-axis display subchannel having a second display optical axis, the second display subchannel comprising second adjustable display optics and a second electronic display, the second adjustable display optics configured to relay an image output on the second display to the second eyepiece via the fourth beamsplitter for viewing by the second eye of the user when the user is wearing the headset frame.

17. The system of claim 14, wherein the second optical channel is substantially the same as the first optical channel.

18. The system of claim 14, wherein the first optical channel and the second optical channel are contained within a common outer housing.
